(19) United States
(12) Patent Application Publication
(10) Pub. No.: US 2026/0053678 A1
(43) Pub. Date: Feb. 26, 2026

(54) DISPOSABLE ABSORBENT INSERT FOR A REUSABLE OUTER SHELL

(71) Applicant: Essity Hygiene and Health Aktiebolag, Göteborg (SE)

(72) Inventors: Helena CORNELIUSSON, Göteborg (SE); Lisa NILSSON, Göteborg (SE); Emelie ADIELS, Göteborg (SE); Yasha ASAYESH, Göteborg (SE)

(73) Assignee: Essity Hygiene and Health Aktiebolag, Göteborg (SE)

(21) Appl. No.: 19/101,634

(22) PCT Filed: Sep. 2, 2022
(86) PCT No.: PCT/EP2022/074474

§ 371 Date: Feb. 6, 2025

(51) Int. Cl. A61F 13/49; A61F 13/514; A61F 13/53; A61F 13/62

(52) U.S. Cl. CPC A61F 13/49004; A61F 13/49017; A61F 13/51474; A61F 13/53; A61F 13/627; A61F 2013/49093

(57) ABSTRACT

A disposable absorbent insert for a reusable outer shell includes a liquid-impermeable backsheet having a backsheet first side edge and a backsheet second side edge, an absorbent core disposed between a liquid-permeable and user facing topsheet and the liquid-impermeable backsheet, and a nonwoven outer cover layer overlaying the backsheet and forming an outer surface of the insert. Each leg cuff includes a side portion of the outer cover layer being folded back on itself along a fold line and dividing the side portion of the outer cover layer in a first part and a second part which first and second parts together form a leg cuff fold, and a plurality of spaced-apart pre-tensioned elastic elements extending in the longitudinal direction inside the leg cuff fold and being attached between the first part and the second part of the leg cuff fold.

### TECHNICAL FIELD

The disclosure relates to an disposable absorbent insert for a reusable outer shell and a wearable absorbent article comprising a reusable shell and a disposable absorbent insert.

### BACKGROUND

Wearable and disposable absorbent articles, for example in the form of diapers, incontinence garments, feminine garments, inserts and the like, are well known. Absorbent articles are used to absorb, distribute and store various types of body exudates while providing a high level of comfort and sense of dryness to the wearer during use.

A conventional disposable absorbent article in the form of a diaper is normally designed with an absorbent core which is sandwiched between a topsheet and a backsheet. The article is arranged along a longitudinal axis and along a transversal axis which extends in a perpendicular direction in relation to the longitudinal axis. Furthermore, the article can be divided into a front portion, a back portion and a crotch portion. The diaper is designed with a waist portion to be enclosing the wearer being integral with the core and further comprising interior leg gathers i.e. standing gathers, and exterior leg gathers to avoid edge leakages from the article.

A disposable absorbent insert is to be used with an outer reusable shell and is normally of a simpler design than a disposable diaper as it is intended to be used with an exterior disposable or reusable shell or cover. There is a general desire to achieve the same level of comfort, absorbent capacity and leakage security in absorbent inserts as with conventional diapers.

### SUMMARY

The present disclosure provides a disposable absorbent insert in accordance with claim 1 and a wearable absorbent article comprising a reusable shell and a disposable absorbent insert according to claim 25. Further embodiments are set out in the dependent claims and in the following description.

Disclosed herein is a disposable absorbent insert for a reusable outer shell, the insert having an extension in the longitudinal direction and the transversal direction, longitudinal side edges and transversal side edges and having a front portion, a back portion and a crotch portion located between the front and the back portions. The disposable absorbent insert comprises a liquid-impermeable backsheet having a backsheet first side edge and a backsheet second side edge, an absorbent core disposed between a liquid-permeable and user facing topsheet and the liquid-impermeable backsheet and a nonwoven outer cover layer overlaying the backsheet and forming an outer surface of the insert. The insert has a pair of leg cuffs extending in the longitudinal direction and being symmetrically arranged along the longitudinal side edges. Each leg cuff comprises a side portion of the outer cover layer being folded back on itself along a fold line extending in the longitudinal direction and dividing the side portion of the outer cover layer in a first part and a second part which first and second parts together form a leg cuff fold. The leg cuffs further comprise a plurality of spaced-apart pre-tensioned elastic elements extending in the longitudinal direction inside the leg cuff fold and being attached between the first part and the second part of the leg cuff fold.

The absorbent core may comprise an absorbent component enclosed by a core cover comprising an upper and a lower side.

The leg cuffs of the insert are capable of preventing body liquids, that may have escaped absorption in the absorbent core of the insert, from soiling the leg surrounding parts of a reusable shell to be used in combination with the disposable insert. This increases the number of uses the outer reusable shell can be used before it needs to be washed, which is a great benefit of a two-piece diaper system comprising a disposable inner part and a reusable outer part.

The leg cuffs of the insert as disclosed herein are well-defined, neat and distinct features which are readily identified by visual inspection of the absorbent insert. In prior art inserts leg elastic is commonly arranged between laterally extending side portions of a topsheet and a backsheet or between side portions of a standing gather material and the backsheet. Such arrangement results in a frilly, floppy non-elasticated side edge portion laterally outside of the elastic elements. When putting on shell with an insert having such non-elasticated side portions, it needs to be ascertained that the side-portions are folded out to avoid that the insert is applied to the wearer with the elastic side portions folded in over the absorbent body, which may lead to poor fit and ultimately to leakage of body fluids out of the insert.

An insert as disclosed herein is skin-friendly and comfortable to wear as all body-contacting surfaces of the article are constituted by soft nonwoven materials. The leg cuffs are particularly soft as the pretensioned elastic elements in the leg cuffs are arranged inside the leg cuff fold, providing the leg cuffs with a folded outer edge. The plurality of spaced-apart pre-tensioned elastic elements cause the nonwoven material in the leg cuff folds to contract and form soft, airy and evenly distributed wrinkles on the surface of the leg cuff. The wrinkled elasticated portion of the leg cuff acts as a cushioning distance means between a wearer's body and the elastic elements.

The side portions of the outer cover layer may extend in the transverse direction outward of the corresponding backsheet side edge.

All or substantially all elastic elements of the plurality of elastic elements in each leg cuff may be arranged in an outer part of the leg cuff which is transversely outside of the liquid impermeable backsheet, i.e. free from the liquid impermeable backsheet.

The nonwoven outer cover layer may be a single layer nonwoven material.

The leg cuffs may be leakage resistant. The nonwoven outer cover layer forming the leg cuffs may be a material providing properties like great breathability and barrier qualities to avoid any leakage and soiling of the reusable outer shell. This is particularly advantageous if there are no standing gathers present on the insert. The nonwoven layer may be made of co-formed lamina of nonwoven materials such as an SMS (spunbond/meltblown/spunbond) nonwoven material or an SS (spunbond/spunbond) nonwoven material. The thermoplastic polymer materials in the nonwoven layer may be polypropylene or bicomponent fibers of polypropylene and polyethylene, or of a combination of such materials. The outer cover layer may also comprise two layers of hydrophobic nonwoven material.

Alternatively, the nonwoven materials used for the outer cover material may include one or more spunbond layers and optionally one or more meltblown layers. The nonwoven material may be made of thermoplastic polymer fibers or filaments. The nonwoven material may be formed by any of a variety of different processes, such as spunbonding, airlaying, meltblowing or bonded carded web formation processes.

The nonwoven materials used may be renewable or recyclable. The nonwoven materials may be biobased.

The leg cuffs are highly breathable, as they are mainly constituted by nonwoven materials. The liquid impermeable backsheet may extend into the leg cuffs in an inner part of each leg cuff by at most 50 % of the width of the leg cuff, such as at most 40 % of the width of the leg cuff or at most 30 % of the width of the leg cuff. The liquid impermeable backsheet may extend into the leg cuffs to a greater part of the width of each leg cuff in smaller sizes of an article than in larger sizes of the article.

The elastic elements of the plurality of elastic elements may be attached between the first and second parts of the leg cuff fold by means of adhesive.

According to one aspect, the first part of the side portion of the outer cover layer may be a lower part of the leg cuff fold, as seen in a direction from the topsheet and the second part of the side portion of the outer cover layer may be an upper part of the leg cuff fold, as seen in a direction from the topsheet. In such an arrangement the outer-facing side of the insert is soft and continuous without any edges, seams or joint. Is such an arrangement the second part of the side portion comprises the longitudinal edge of the outer cover layer, and the longitudinal edge of the second part of the side portion is an inner end which may be secured between the topsheet and the backsheet.

The plurality of elastic elements in each leg cuff may be constituted by at least 3 elastic elements, preferably at least 4 elastic elements. The elastic elements may be spaced apart in the transverse direction by from 1 mm to 8 mm, preferably from 3 mm to 8 mm.

The elastic elements may be elastic threads, strings or elastic bands, preferably elastic threads.

One or more elastic elements being arranged in an inner part of the leg cuff fold may have a greater tensile resistance than one or more elastic elements being arranged in an outer part of the leg cuff fold, and one or more elastic elements in the inner part of the leg cuff fold may have a linear density (dtex) which is higher than a linear density of the one or more elastic elements being arranged in the outer portion of the leg cuff fold.

The leg cuff fold may extend along the entire longitudinal length of the insert.

The plurality of elastic elements may be arranged at least along the entire longitudinal length of the crotch area, and the longitudinal length of the plurality of elastic elements is less than the total longitudinal length of the insert. By such, the folded side portion of the nonwoven outer cover layer is only elasticized at the part of the insert intended to be arranged adjacent the wearers legs during use. The leg cuff fold partly lacks elastic elements in the front portion and the back portion, thereby providing the insert with a softer front and back which will adapt better to the waist of the wearer once placed in the outer reusable shell and worn.

Furthermore, the insert may comprise a first longitudinal extending standing gathers and a second longitudinal extending standing gather. The standing gathers are arranged transversally inside the leg cuffs, towards the longitudinal center line of the insert, and are arranged to be at least partly raised during use of the insert. The standing gathers are attached to the topsheet. The standing gathers further improve the leakage security of the absorbent insert, in addition to the leg cuffs.

The insert may have a pair of nonwoven barrier cuff strips extending in the longitudinal direction and being symmetrically arranged with one barrier cuff strip on each side of the longitudinal centre line. Each nonwoven barrier cuff strip may comprise a standing gather portion forming the first and second standing gather respectively, each standing gather being defined between a free standing gather edge extending in the longitudinal direction and a standing gather hinge line extending in the longitudinal direction. The barrier cuff strip is attached to the topsheet along the standing gather hinge line.

The nonwoven material in the nonwoven barrier strip may be the same as described in conjunction with the outer cover layer herein. The nonwoven material in the nonwoven barrier strip may be different from the nonwoven material of the outer cover material from which the leg cuff fold is formed.

The standing gathers may have one or more pre-tensioned elastic elements arranged along the free standing gather edge.

The nonwoven barrier cuff strip may comprise a leg cuff portion and the leg cuff portion of the nonwoven barrier cuff strip being defined between the standing gather hinge line and an outer leg cuff edge of the nonwoven barrier cuff strip which outer leg cuff edge of the nonwoven barrier cuff strip is parallel to the fold line in the leg cuff fold, the leg cuff portion of the nonwoven barrier cuff strip overlying at least one elastic element of the plurality of elastic elements.

Each leg cuff may be arranged outward of the standing gather portions of the corresponding barrier cuff strip.

The leg cuff fold may have a free edge defined by the fold line and a leg cuff width in the transverse direction between the fold line in the leg cuff fold and the standing gather hinge line.

The leg cuff portion of each nonwoven barrier cuff strip may be attached to the corresponding leg cuff fold with the leg cuff portion of the nonwoven barrier cuff strip overlaying all, or substantially all of the plurality of elastic elements in the leg cuff fold.

In such an arrangement, the leg cuffs comprise the leg cuff portion of the nonwoven barrier cuff strip at the inner wearer facing side of the article, which forms an additional gatherable cushioning layer of the leg cuff. Thus, under the influence of the contractive forces from the elastic elements which are distributed over the width of each leg cuff, the leg cuff portion of the nonwoven barrier cuff strip together with the nonwoven material in the leg cuff fold form a soft, bulky, yet highly body conforming leg cuff.

In an insert as disclosed herein, the leg cuff portion of the nonwoven barrier cuff strip may be attached to the leg cuff fold with an adhesive being uniformly distributed over the attachment area between the nonwoven barrier cuff strip and the leg cuff fold or with uniformly distributed thermo-bonds or ultrasonic bonds. Accordingly, the adhesive is uniformly distributed over an attachment area between the leg cuff portion of the nonwoven barrier cuff strip and the upper part of the side portion of the outer cover layer. The adhesive may be applied by any suitable technique, such as slot-coating or spray-coating.

The outer leg cuff edge of the nonwoven barrier cuff strip may be arranged at or inward of the fold line in the leg cuff fold as seen in the transverse direction.

The leg cuff portion of the nonwoven barrier cuff strip may be attached to the second part of the side portion of the outer cover layer with an adhesive, the adhesive being uniformly distributed over an attachment area between the leg cuff portion of the nonwoven barrier cuff strip and the second part of the side portion of the outer cover layer.

The width of each leg cuff is in the range of from 20 millimetres to 60 millimetres and wherein the elastic elements of the plurality of elastic elements in each leg cuff are distributed over an area of the leg cuff having a width in the transverse direction which is at least 40 % of the width of the leg cuff, preferably at least 50 % of the width of the leg cuff.

The nonwoven barrier cuff strip may be a single layer nonwoven material.

The first part of the side portion of the outer cover layer may be contiguous with a central part of the outer cover layer, which central part of the outer cover layer overlies the backsheet and wherein the second part of the side portion of the outer cover layer is the folded-over part of the side portion of the outer cover layer.

The insert may be provided with fastener elements to fasten the insert to the reusable outer shell. The reusable outer shell may also be provided with corresponding fastener elements to facilitate fastening of the insert. Suitable fastener elements may for example include hook or loop materials, push buttons and tape.

The fastener elements may be a strip of loop material attached to a garment facing side of the outer cover layer near the transverse side edge of the front portion and back portion respectively of the insert.

Fastener elements may be a hook member comprising any hook-type material with a hook surface structure or a hook attachment zone comprising any loop surface structure, i.e. the system known as a VELCRO® system. The “hooks” may have many different shapes which are adapted to engage with a loop part of the hook and loop fastening system. known as a VELCRO® system.

The strip of loop material may extend over 50-100 % of the transversal side of the insert. The longitudinal extension of the strip of loop material may be 10 to 50 mm, such as 10 to 35 mm. The longitudinal extension of the strip of loop material may be greater in the back portion than in the front portion of the insert. The strip of loop material may have the same free transversal side edge as materials of the topsheet and the backsheet. The strip of loop material may have the same free transversal side edge as the insert. The insert with the strips of loop material attached to the backsheet may further comprise any other feature of the insert as disclosed above and herein.

The strips of loop material on the garment facing side of the backsheet provide improved attachment of the insert into a reusable shell provided with hook patches at an inside of a front and back portion thereof such as disclosed herein. An advantage of an insert having a strip of loop material in the front and back portion thereof with the same free transversal side edges as the insert itself is that it will provide improved and close attachment of the insert, when in use, in a reusable shell.

Alternatively, the outer cover layer itself may be of a material which facilitates fastening of the insert to the reusable outer shell, i.e. a material which provides for secure attachment of the insert to hook material elements provided on the user-facing side of the outer shell.

The disclosure concerns a disposable absorbent insert for an outer reusable shell. The insert thus lacks side flaps arranged at the waist edges of the front portion and side flaps arranged at the waist edges of the back portion. Such side flaps are present on disposable diapers and provided with fasteners for fastening the disposable diaper in a pant-like fashion around the waist of a wearer. The disposable absorbent insert is not intended to be fastened to the waist of a wearer without an outer shell, and thus lacks any such fastening means.

Moreover, the insert lacks a waist belt. Such waist belts are present on conventional pant type disposable diapers and form a continuous waistband around the body of a wearer.

The insert may have a rectangular shape. The absorbent core may have a rectangular shape.

The insert may be provided with a waist elastic member at the traversal front edge of the front portion and/or traversal rear edge at the back portion of the insert. The waist elastic member may comprise an elasticized material or elastic threads extending in transversal direction. The waist elastic member may improve fit around the waist of the wearer.

The insert may be provided with a waist gasketing element in the form of a barrier pocket in the front portion and/or back portion of the insert. The barrier pocket is attached to the insert along its longitudinal edges and front or back edge respectively, such as to open up towards the crotch portion and thereby forming a pocket. Such waist gasketing elements may improve leakage security around the waist of the wearer.

This disclosure also concerns a wearable absorbent article comprising a reusable outer shell, to be worn by a wearer, and a disposable absorbent insert as described herein. A reusable outer shell may be made of garment-like fabric, be underwear-like and may be washable.

A wearable absorbent article comprising a reusable outer shell, to be worn by a wearer, may comprise a disposable absorbent insert comprising a strip of loop material attached to the garment facing side of the backsheet near the transverse side edge of the front and back portions respectively of the insert, as disclosed above.

### BRIEF DESCRIPTION OF THE DRAWINGS

The disclosure will be described in greater detail below with reference to the figures shown in the appended drawings, wherein:

FIG. 1 shows a planar view of a stretched-out absorbent article in the form of an insert, seen from the side which is intended to be facing a wearer during use;

FIG. 2 shows a cross-section through the insert in FIG. 1, taken along the line II-II in FIG. 1;

FIG. 3 shows a wearable absorbent article comprising a reusable shell and a disposable insert;

FIG. 4 shows another planar view of a stretched-out absorbent article in the form of an insert, seen from the side which is intended to be facing a wearer during use;

FIG. 5 shows a cross-section through the insert in FIG. 3, taken along the line II-II in FIG. 3;

FIG. 6 shows a detail of a leg cuff as shown in FIGS. 4 and 5; and

FIG. 7 shows an alternative cross-section through the diaper in FIG. 1, taken along the line II-II in FIG. 4.

### DETAILED DESCRIPTION OF THE DRAWINGS

It is to be understood by one of ordinary skill in the art that the following is a description of exemplary embodiments only, and is not intended as limiting the broader aspects of the present disclosure.

Different aspects of the absorbent insert as disclosed herein will be described more fully hereinafter with reference to the enclosed drawings. The embodiments disclosed herein should not be construed as being limiting to the claimed absorbent insert.

It is to be understood that the drawings are schematic and that individual components, such as layers of material are not necessarily drawn to scale.

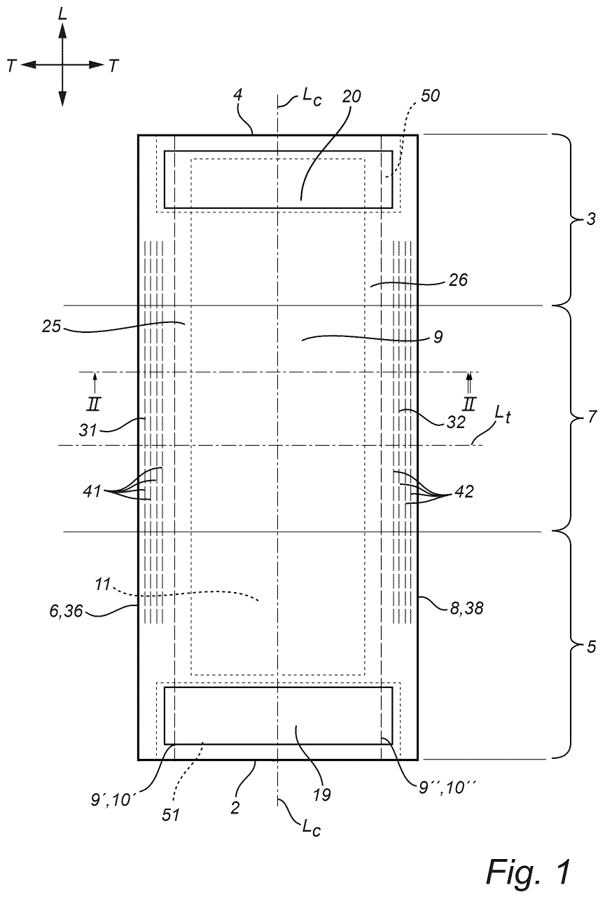

A disposable absorbent insert 1 is shown in FIG. 1 in an unfolded and flat condition with all elastic elements and components in an extended state. FIG. 1 shows the insert 1 from an inner, wearer-facing surface. Any measurements of distances and dimensions are made with the insert in the fully stretched-out, non-gathered condition as shown in FIG. 1.

The insert 1 has a longitudinal direction L and a transverse direction T.

The insert 1 has a first waist edge 2 and a second waist edge 4 and first and second side edges 6, 8. The first waist edge 2 of the insert 1 which is shown in FIG. 1 is a rear waist edge and the second waist edge 4 of the absorbent insert 1 is a front waist edge. The first and second waist edges 2, 4 extend in a transverse direction T of the absorbent insert 1 and the side edges 6, 8 extend in a longitudinal direction L of the absorbent insert 1. The absorbent insert 1 may be divided in the longitudinal direction L into a from portion 3, a rear portion 5 and a crotch portion 7 located between the front portion 3 and the rear portion 5.

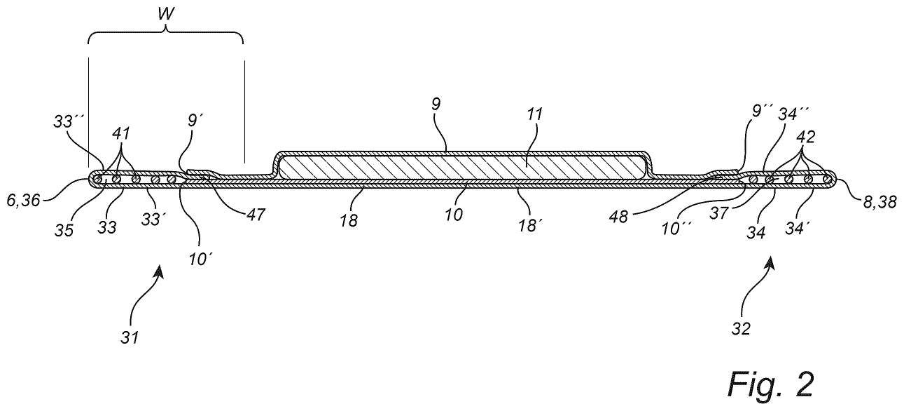

The insert 1 comprises a liquid permeable topsheet 9 and a liquid impermeable backsheet 10. An absorbent core 11 is disposed between the topsheet 9 and the backsheet 10. The topsheet 9 has a topsheet first side edge 9′ and a topsheet second side edge 9″ and the backsheet 10 has a backsheet first side edge 10′ and a backsheet second side edge 10″, as indicated in FIGS. 1 and 2.

The insert 1 further comprises a nonwoven outer cover layer 18 overlaying the backsheet 10 and forming an outer surface of the insert 1.

The insert 1 comprises a pair of leg cuffs 31, 32 extending in the longitudinal direction L and being symmetrically arranged along the first and second side edges 6, 8.

As shown in FIG. 2, the leg cuffs 31, 32 each comprises a side portion 33, 34 of the outer cover layer which extends in the transverse direction outward of the corresponding backsheet side edge 10′, 10″, the side portion 33, 34 of the outer cover layer 18 being folded back on itself along a fold line 36, 38 extending in the longitudinal direction L and dividing the side portion 33, 34 of the outer cover layer 18 in a first part 33′, 34′ and a second part 33″, 34″ which first and second parts 33′, 33″; 34′, 34″ together form a leg cuff fold 35, 37 having a free edge defined by the fold line 36, 38 and a leg cuff width W being defined in the transverse direction T between the free edge of the leg cuff fold line 36, 38 and the inner ends 47, 48 of the side portion 33, 34.

The first parts 33′, 34′ of the side portions 33, 34 of the outer cover layer 18 are shown in FIG. 2 to be contiguous with a central part 18′ of the outer cover layer 18, which central part 18′ of the outer cover layer 18 overlies the backsheet 10. The second parts 33″, 34″ of the side portions 33, 34 of the outer cover layer 18 are the folded over parts of the side portions 33, 34 of the outer cover layer 18.

In the embodiment shown in the FIGS. 1-6, the first parts 33′, 34′ of the side portions 33, 34 of the outer cover layer 18 constitute lower parts of the leg cuff folds 35, 37, as seen in a direction from the topsheet 9 and the second parts 33″, 34″ of the side portions 33, 34 of the outer cover layer 18 constitute upper parts of the leg cuff folds 35, 37, as seen in a direction from the topsheet 9. The upper parts of the leg cuff folds 35, 37 are folded back on top of the lower parts of the leg cuff folds 35, 37 and are secured between the topsheet 9 and the backsheet 10. Hence, the topsheet 9 and the backsheet 10 are directly connected to each other along side edges of the absorbent core 11 and directly connected to the folded-in parts of the outer cover layer which are secured between the topsheet 9 and the backsheet 10, as seen in FIG. 2. The folded-in parts of the outer cover layer comprise the inner ends 47, 48. The garment-facing side of the backsheet is directly attached to the central part of the outer cover sheet.

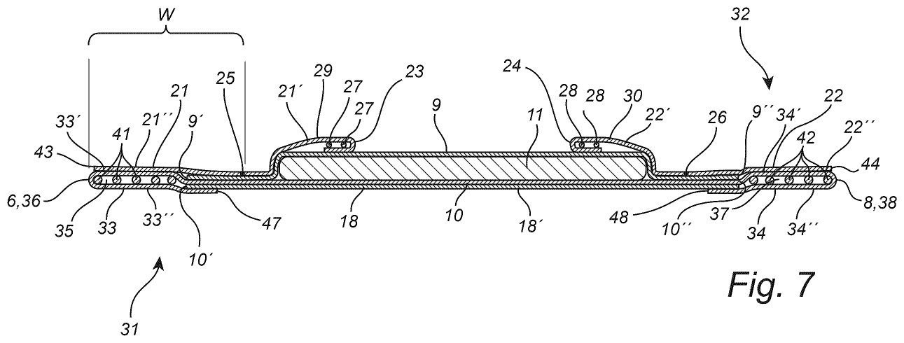

As set out herein, the side portions 33, 34 of the outer cover layer 18 may alternatively be folded such that the first parts 33′, 34′of the side portions 33, 34 of the outer cover layer 18, which are contiguous with the central part 18′ of the outer cover layer 18 constitute upper parts of the leg cuff folds 35, 37 as seen in a direction from the topsheet and the folded-back second parts 33″, 34″ constitute lower parts of the leg cuff folds 35, 37, as seen in a direction from the topsheet 9. In this configuration, as shown in FIG. 7, the side portions 33, 34 of the outer cover layers 18 are folded back on themselves on the outer garment-facing side of the insert 1.

A plurality of spaced-apart pre-tensioned elastic elements 41, 42 are arranged in each leg cuff fold 35, 37. The elastic elements 41, 42 extend in the longitudinal direction L inside the leg cuff fold 35, 37 and are attached, e.g., by means of adhesive, between the first part 33′, 34′ and the second part 33″, 34″ of the leg cuff fold 35, 37.

The width of the leg cuffs 31, 32 may be in the range of from 20 millimetres to 60 millimetres and the elastic elements of the plurality of elastic elements 41, 42 may be distributed over an area of each leg cuff having a width which is at least 40 % of the width of the leg cuff, preferably at least 50 % of the width of the leg cuff. The width of the elasticated portion of the leg cuffs 31, 32 may be in the range of from 20 millimetres to 40 millimetres.

The elastic elements of the plurality of elastic elements 41, 42 in each leg cuff 31, 32 may suitably be attached between the first and second parts 33′, 34′; 33″, 34″ of the leg cuff fold 35, 37 by means of adhesive. Adhesive may be applied to the elastic elements, and/or to one or both of the first and second parts 33, 34 of the leg cuff fold 35, 37. Alternatively, the elastic elements may be anchored in the leg cuff fold 35, 37 by means of heat welding or ultrasonic welding, e.g., as disclosed in WO2021/037352 A1.

The elastic elements may be elastic threads, bands, strings, etc. All elastic elements may be of the same type and having the same elastic properties. Alternatively, one or more elastic elements being arranged in an outer part of the leg cuff fold 35, 37 may have a greater tensile resistance than one or more elastic elements being arranged in an inner part of the leg cuff fold 35, 37.

The one or more elastic elements in the outer part of the leg cuff fold 35, 37 may have a linear density (dtex) which is higher than the linear density of the one or more elastic elements being arranged in the inner portion of the leg cuff fold 35, 37.

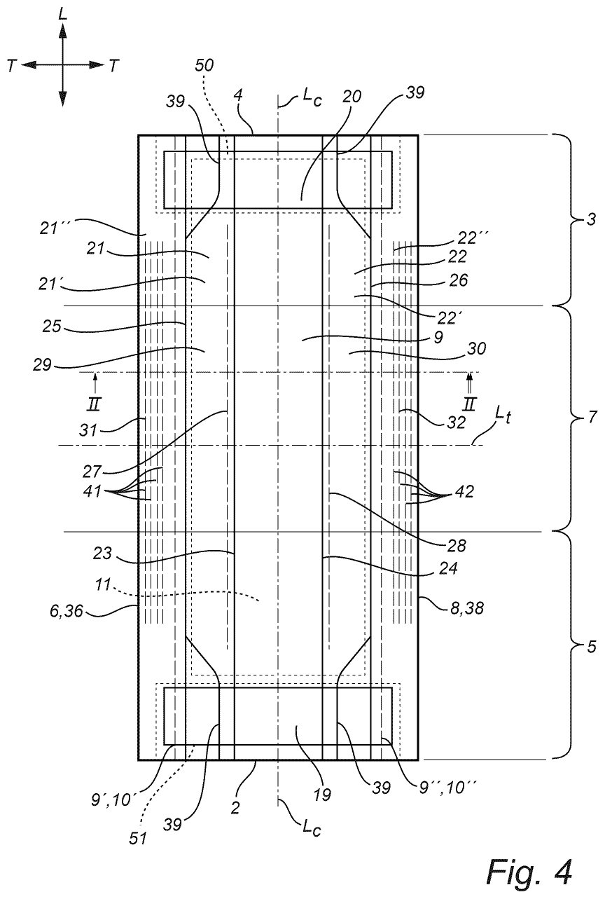

The absorbent insert 1 in FIGS. 1 and 4 further includes an optional waist elastic member 19. The waist elastic member 19 is located at the rear waist edge 2 and extends in the lateral direction along only a part of the rear waist edge 2. Any type of waist elastic may be used, as known in the art. Furthermore, the absorbent insert 1 may include waist elastic member 20 also at the front portion 3 of the absorbent insert 1.

Fastener elements, in form of a strip of loop material 50, 51 is attached to the garment-facing side of the backsheet 18 near the transversal side 2, 4 edge of the front 3 and back 5 portions respectively of the insert 1, as shown in FIGS. 1, 3 and 4. The strip of loop material 50, 51 extends over 50-100 % of the transverse side of the insert 1. The longitudinal extension of each strip of loop material is 10 to 50 mm. The longitudinal extension of the strip of loop material 50, 51 may be greater in the back portion 5 than in the front portion 3 of the insert 1.

Continuous production of absorbent articles is well known in the field. The method of producing a disposable absorbent insert 1 for use with a reusable outer shell 100 may comprise: conveying a continuous precursor of absorbent inserts 1 having an extension in the longitudinal direction and the transversal direction, the precursor having a continuous topsheet material 9 and a continuous liquid-impermeable backsheet material 10 and having multiple absorbent cores 11 distributed regularly between the topsheet 9 and backsheet 10 materials. The precursor also having a continuous outer cover layer material 18. The method as disclosed herein then further regularly provides a strip of loop material 50, 51 to an outer facing side of the outer cover layer material 18 so that it bridges an outline of two nearby and interconnected inserts 1 each comprising an absorbent core 11, and cutting the strip of loop material 50, 51 and the topsheet 9, the backsheet 10 and the outer cover layer 18 materials in one cutting operation to separate the connected inserts 1 to thereby obtain single inserts 1 having a strip of loop material 50, 51 near the transversal side edge 2, 4 of the front 3 and the back 5 portions respectively of the inserts 1.

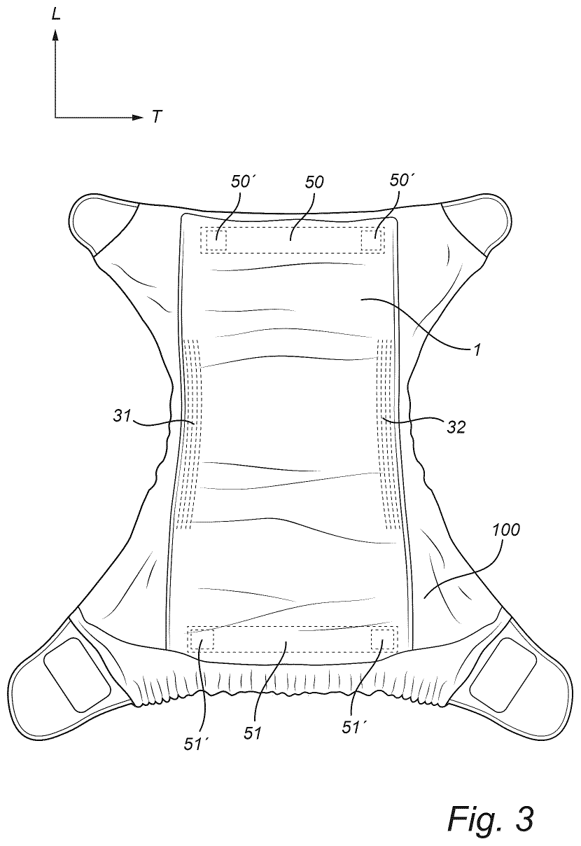

FIG. 3 discloses the absorbent insert 1 in an outer reusable shell 2. The reusable shell 2 has longitudinal and transversal side edges and an inner surface and an outer surface. FIG. 3 is an illustration of the reusable shell 100 as seen from the inner surface facing the user when worn. The reusable outer shell is provided with complementary fastener elements 50′, 51′.

The reusable outer shell 2 is an outer shell that is capable of being reused multiple times. The reusable outer shell may be a washable outer shell, capable of being washed multiple times without essential shrinkage or other damage. The outer shell may be made from a washable material such as a washable fabric. The outer shell may be made of one or more of the following materials: polyester, viscose, polyamide and/or cotton. The outer shell may be a stretchable outer shell, i.e. being made of a stretchable material. The outer shell may be an elastic shell, being elastic in the longitudinal direction L and/or the transverse direction T of the outer shell.

In one example to releasably attach the absorbent insert 1 to the outer shell 100, the outer shell 100 comprises a front hook member 50′ and a rear hook member 51′ arranged on the inside surface of the outer shell 100 and to be attached to the front and rear loop members or strips of loop material 50, 51 attached to the garment facing side of the outer cover layer 18 of the insert 1.

As further seen in FIG. 3, the broad, elasticated leg cuffs 31, 32 are bulky and shape stable and automatically assumes a correct position when the outer shell and insert is applied to a wearer.

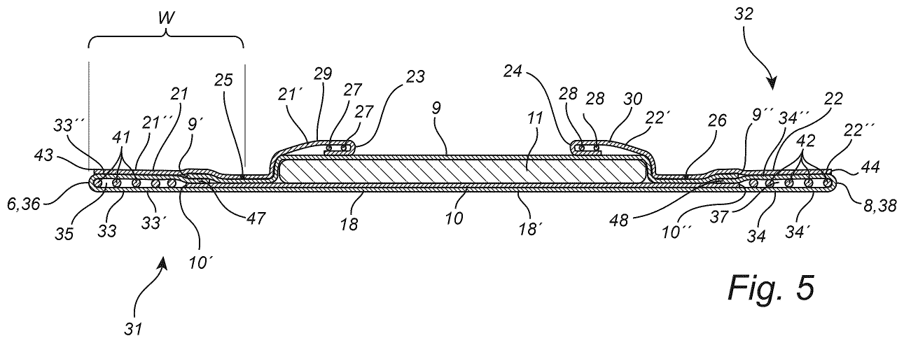

As seen in FIG. 4 and FIG. 5, an insert as seen in FIGS. 1, 2 and 3 may further be provided with standing gathers 29, 30. A pair of nonwoven barrier cuff strips 21, 22 are arranged extending in the longitudinal direction L and are symmetrically arranged with one barrier cuff strip 21, 22 on each side of a longitudinal centre line Lc, perpendicular to a transverse centre line Lt, each nonwoven barrier cuff strip 21, 22 comprising a standing gather portion 21′, 22′. The standing gather portion 21′, 22′ of the nonwoven barrier cuff strip 21, 22 is defined between a free standing gather edge 23, 24 extending in the longitudinal direction L and a standing gather hinge line 25, 26 extending in the longitudinal direction L. A pair of raised elasticated standing gathers 29, 30 comprising the standing gather portions 21′, 22′ of the barrier cuff strips 21, 22 are arranged on the topsheet 9. Hence, the standing gather portions 21′, 22′ of the barrier cuff strips 21, 22 form parts of the standing gathers 29, 30.

Each barrier cuff strip 21, 22 is directly attached to the topsheet 9 along the standing gather hinge line 25, 26 and has one or more pre-tensioned elastic elements 27, 28 arranged along the free standing gather edge 23, 24. The elastic element or elements may be elastic strings, threads or bands which are applied spaced apart along the free standing gather edge 23, 24. In an absorbent insert as disclosed herein the number of elastic elements 27, 28 which are disposed along the standing gather edge, 23, 24 is typically 1, 2 or 3. The elastic elements 27, 28 are typically disposed parallel to each other with a spacing of from 2 to 5 millimetres, with an outermost elastic element arranged at the free standing gather edge 23, 24.

Each leg cuff 31, 32 being arranged outward of the standing gather portion 21′, 22′ of the corresponding barrier cuff strip 21, 22.

Each leg cuff 31, 32 further comprises a leg cuff portion 21″, 22″ of the corresponding nonwoven barrier strip 21, 22. The leg cuff portion 21″, 22″ of the nonwoven barrier cuff strip 21, 22 is defined between the standing gather hinge line 25, 26 and an outer leg cuff edge 43, 44 of the nonwoven barrier cuff strip 21, 22.

The outer leg cuff edge 43, 44 of the nonwoven barrier cuff strip 21, 22 is parallel to the fold-line 36, 38 in the leg cuff fold 35, 37 and overlies at least one elastic element of the plurality of elastic elements 41, 42. In the embodiment shown in the figures, the leg cuff portions 21″, 22″ of the nonwoven barrier strips 21, 22 overlie all elastic elements of the plurality of elastic elements 41, 42 in the leg cuff folds 35, 37 and the outer leg cuff edges 43, 44 of the leg cuff portions 21″, 22″ coincide with the fold lines 36, 38 in the side portions 33, 34 of the outer cover layer 18. It is to be understood that the outer leg cuff edges 43, 44 of the leg cuff portions 21″, 22″ may not coincide with the fold lines 36, 38 and may be arranged inward of the fold lines 36, 38 in the side portions 33, 34 of the outer cover layer 18.

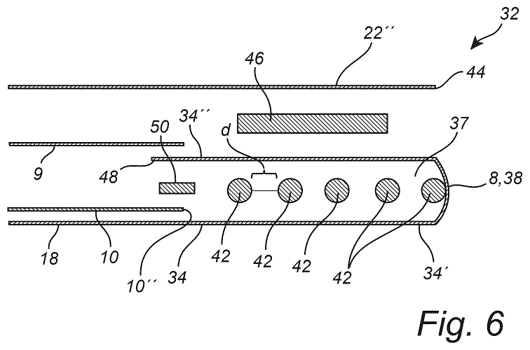

FIG. 6 shows a simplified detail of a portion of the leg cuff 32 of FIG. 5 which is arranged along the second side edge. A plurality of elastic elements 42, represented by five individual elastic elements are arranged in the leg cuff fold 37 with a spacing d between the individual elastic elements. The elastic elements are shown with equal spacing between all elastic elements. However, the spacing may be different in different parts of the leg cuff 32. By way of example, the spacing between the individual elastic elements may be smaller in an outer part of the leg cuff 32 which is close to the fold line 38 than in an inner part of the leg cuff 32 which is closer to the standing gather hinge line 26.

The leg cuff portion 22″ of the nonwoven barrier cuff strip 22 overlies all elastic elements of the plurality of elastic elements 42 in the leg cuff fold 37 but is only attached to the second, upper, part 34″ of the leg cuff fold 37 in an area of the second part 34″ of the leg cuff fold 37 corresponding to the location of the 4 elastic elements of the plurality of elastic elements 42 which are arranged at a distance from the fold line 38. The leg cuff portion 22″ of the nonwoven barrier cuff strip 22 is attached to the second part 34″ of the leg cuff fold 37 by means of adhesive 46, leaving the outermost part of the leg cuff portion 22″ of the nonwoven barrier cuff strip 22 free from attachments to the leg cuff fold 37. In this manner, no adhesive is arranged immediately adjacent to the side edge of the absorbent insert 1. The adhesive attachment between the leg cuff fold and the leg cuff portion 22″ of the nonwoven barrier cuff strip 22 may increase the structural stability of the leg cuff 32 by increasing the bending resistance of the leg cuff. It is to be understood that the adhesive area 46 may extend all the way to the fold line 38 in the leg cuff fold 37.

As shown in FIG. 6, which is a highly schematic, exploded view of the leg cuff 32, the outermost elastic element of the plurality of elastic elements 42 is positioned at the fold line 38 such that no excess material is present outward of the outermost elastic element. This may be an ideal positioning of the outermost elastic element. However, due to production tolerances, the positioning of the outermost elastic element in an actual absorbent insert 1 may deviate somewhat from the position shown in FIG. 6.

As disclosed herein, the leg cuff portion 22″ of the nonwoven barrier cuff strip 22 may be attached to the leg cuff fold 37 with an adhesive which has been uniformly distributed over the attachment area between the barrier cuff strip 22 and the upper part 34″ of the leg cuff fold 37, creating a uniform attachment between the leg cuff portion 22″ of the nonwoven barrier cuff strip 22 and the leg cuff fold 37 so that the contractive forces from the spaced-apart pre-tensioned elastic elements 42 in the leg cuff fold 37 are transferred to the leg cuff portion 22″ of the nonwoven barrier cuff strip 22 in a substantially equal way over the whole attachment area. Thereby, when the elastic elements contract, the leg cuff portion 22″ of the nonwoven barrier cuff strip 22 gathers together with the material in the leg cuff fold, 37 and forms soft corrugations in the leg cuff 32 in a controlled and neat manner.

As also seen in FIG. 6, the second part 34″ of the leg cuff fold 37 is secured to the backsheet 10 along the backsheet side edge 10″ at an inner end 48 of the second part 34″ of the leg cuff fold 37. The inner end 48 is also arranged underneath the topsheet 9. As seen in FIG. 2 and FIG. 6, the inner ends 47, 48 of the second parts 33″, 34″ of the side portions 33, 34 are inserted between the topsheet 9 and the backsheet 10 and are secured between the topsheet 9 and the backsheet 10. The attachment is made by means of adhesive 50. However, it is to be understood that the leg cuff portion 22″ of the nonwoven barrier strip 22 may alternatively be attached by means of thermo-welding or ultrasonic welding. In the embodiment shown in FIG. 3, all elastic elements of the plurality of elastic elements 42 are arranged in a portion of the leg cuff fold 37 where the backsheet 10 is absent. Although, this may be a desired arrangement providing maximum breathability to the leg cuff 32, the backsheet 10 may alternatively extend into the leg cuff 32 such that it overlaps with an innermost elastic element of the plurality of elastic elements 42.

In the configuration shown in FIG. 7, the leg cuffs 31, 32 each comprises a side portion 33, 34 of the outer cover layer which extends in the transverse direction outward of the corresponding backsheet side edge 10′, 10″, the side portion 33, 34 of the outer cover layer 18 being folded back on itself along a fold line 36, 38 extending in the longitudinal direction L and dividing the side portion 33, 34 of the outer cover layer 18 in a first part 33′, 34′ and a second part 33″, 34″ which first and second parts 33′, 33″; 34′, 34″ together form a leg cuff fold 35, 37 having a free edge defined by the fold line 36, 38 and a leg cuff width W being defined in the transverse direction T between the free edge of the leg cuff fold line 36, 38 and the standing gather hinge line 25, 26. The first parts 33′, 34′ of the side portions 33, 34 of the outer cover layer 18 are contiguous with a central part 18′ of the outer cover layer 18, which central part 18′ of the outer cover layer 18 overlies the backsheet 10.

As shown in FIG. 7, the first parts 33′, 34′ of the side portions 33, 34 of the outer cover layer 18 constitute upper parts of the leg cuff folds 35, 37, as seen in a direction from the topsheet 9 and the second parts 33″, 34″ of the side portions 33, 34 of the outer cover layer 18 constitute lower parts of the leg cuff folds 35, 37, as seen in a direction from the topsheet 9. The lower parts of the leg cuff folds 35, 37 are folded back underneath of the upper part of the leg cuff folds 35, 37 and is attached to the outer cover layer 18.

The following paragraphs describes the absorbent core, topsheet and backsheet as suitable in all examples described above.

The absorbent core 11 is disposed between the topsheet 9 and the backsheet 10 and is arranged to absorb liquid, such as urine or other body fluids, which has passed through the topsheet 9. The absorbent core 11 may be a single-layer structure or may be a layered structure. The absorbent core 11 may comprise any conventional material suitable for absorbing discharged bodily wastes, such as cellulosic fluff pulp, tissue layers, highly absorbent polymers (super absorbents), absorbent foam materials including hydrogel-foam material, nonwoven materials, or the like. The absorbent core may comprise non-absorbent components such as stiffening elements, shaping elements, binders, etc. Various types of liquid-receiving and liquid distribution materials can also be included in the core.

The absorbent core 11 may be a three-dimensionally shaped core with different amounts of absorbent material in different parts of the core. The core may be provided with features such as embossings, fluid acquisition cavities and fluid distribution channels. Properties of the absorbent core such as density, amount of absorbent material and absorbent capacity may also be varied in different portions of the absorbent core. The absorbent core 11 may comprise a core cover, sandwiching absorbent material between an upper core cover layer and a lower core cover layer. Such core cover may be a tissue material or a nonwoven material and is provided in addition to the topsheet 9 and the backsheet 10.

The liquid permeable topsheet 9 may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet of absorbed body fluid.

The liquid impermeable backsheet 10 may be any conventional liquid impermeable material such as a thin plastic film, e.g. a polyethylene or polypropylene film. The backsheet material may be breathable to allow vapour to escape from the absorbent body, while still preventing liquids from passing through the backsheet material.

1. A disposable absorbent insert for a reusable outer shell, the insert having an extension in the longitudinal direction and the transversal direction, longitudinal side edges and transversal side edges and having a front portion, a back portion and a crotch portion located between the front and the back portions, and comprising
a liquid-impermeable backsheet having a backsheet first side edge and a backsheet second side edge;
an absorbent core disposed between a liquid-permeable and user facing topsheet and the liquid-impermeable backsheet,
a nonwoven outer cover layer overlaying the backsheet and forming an outer surface of the insert;
wherein the insert has
a pair of leg cuffs extending in the longitudinal direction and being symmetrically arranged along the longitudinal side edges, and wherein
each leg cuff comprises:
a side portion of the outer cover layer being folded back on itself along a fold line extending in the longitudinal direction and dividing the side portion of the outer cover layer in a first part and a second part which first and second parts together form a leg cuff fold,
a plurality of spaced-apart pre-tensioned elastic elements extending in the longitudinal direction inside the leg cuff fold and being attached between the first part and the second part of the leg cuff fold.

2. A disposable absorbent insert according to claim 1, wherein the side portions of the outer cover layer extends in the transverse direction outward of the corresponding backsheet side edge.

3. A disposable absorbent insert according to claim 1, wherein all or substantially all elastic elements of the plurality of elastic elements in each leg cuff are arranged in an outer part of the leg cuff transversely outside of the liquid impermeable backsheet.

4. A disposable absorbent insert according to claim 1, wherein the elastic elements of the plurality of elastic elements are attached between the first and second parts of the leg cuff fold by means of adhesive.

5. A disposable absorbent insert according to claim 1, wherein the first part of the side portion of the outer cover layer is contiguous with a central part of the outer cover layer, which central part of the outer cover layer overlies the backsheet and wherein the second part of the side portion of the outer cover layer is the folded-over part of the side portion of the outer cover layer.

6. A disposable absorbent insert according to claim 1, wherein the plurality of elastic elements in each leg cuff are constituted by at least 3 elastic elements.

7. A disposable absorbent insert according to claim 1, wherein the elastic elements are spaced apart in the transverse direction by from 1 mm to 8 mm.

8. A disposable absorbent insert according to claim 1, wherein the plurality of elastic elements is arranged at least along the entire longitudinal length of the crotch area, and the longitudinal length of the plurality of elastic elements is less than the total longitudinal length of the insert.

9. A disposable absorbent insert according to claim 1 wherein the first part of the side portion of the outer cover layer is a lower part of the leg cuff fold, as seen in a direction from the topsheet and the second part of the side portion of the outer cover layer is an upper part of the leg cuff fold, as seen in a direction from the topsheet.

10. A disposable absorbent insert according to claim 9, wherein the second part of the side portion comprises the longitudinal edge of the outer cover layer, and wherein the longitudinal edge of the second part of the side portion is an inner end secured between the topsheet and the backsheet.

11. A disposable absorbent insert according to claim 1, wherein the insert comprising a first longitudinal extending standing gathers and a second longitudinal extending standing gather.

12. A disposable absorbent insert according to claim 11, wherein the standing gathers are attached to the topsheet.

13. A disposable absorbent insert according to claim 11, the insert further comprises a pair of nonwoven barrier cuff strips extending in the longitudinal direction and being symmetrically arranged with one barrier cuff strip on each side of a longitudinal centre line, each nonwoven barrier cuff strip comprising a standing gather portion forming the first and second standing gather respectively, each standing gather being defined between a free standing gather edge extending in the longitudinal direction and a standing gather hinge line extending in the longitudinal direction and wherein the barrier cuff strip is attached to the topsheet along the standing gather hinge line.

14. A disposable absorbent insert according to claim 13, wherein the standing gathers having one or more pre-tensioned elastic elements arranged along the free standing gather edge(23, 24).

15. A disposable absorbent insert according to claim 13, wherein the nonwoven barrier cuff strip, comprises a leg cuff portion being defined between the standing gather hinge line and an outer leg cuff edge of the nonwoven barrier cuff strip which outer leg cuff edge of the nonwoven barrier cuff strip is parallel to the fold line in the leg cuff fold, the leg cuff portion of the nonwoven barrier cuff strip overlying at least one elastic element of the plurality of elastic elements.

16. A disposable absorbent insert according to claim 11, the leg cuff fold having a free edge defined by the fold line and a leg cuff width in the transverse direction between the fold line in the leg cuff fold and the standing gather hinge line.

17. A disposable absorbent insert according to claim 15, wherein the leg cuff portion of each nonwoven barrier cuff strip is attached to the corresponding leg cuff fold with the leg cuff portion of the nonwoven barrier cuff strip overlaying all, or substantially all of the plurality of elastic elements in the leg cuff fold.

18. A disposable absorbent insert according to claim 13, wherein the nonwoven barrier cuff strip is a single layer nonwoven material.

19. A disposable absorbent insert according to claim 4, wherein the nonwoven outer cover layer is a single layer nonwoven material.

20. A disposable absorbent insert according to claim 1, wherein the insert is provided with fastener elements to fasten the insert to a reusable outer shell.

21. A disposable absorbent insert according to claim 20, wherein the fastener elements is a strip of loop material attached to a garment-facing side of the outer cover layer near the transversal side edge of the front portion and back portion respectively of the insert.

22. A disposable absorbent insert according to claim 1, wherein the insert lacks side flaps arranged at the waist edges of the front portion and lacks side flaps arranged at the waist edges of the back portion.

23. A disposable absorbent insert according to claim 1, wherein the insert lacks a waist belt.

24. A disposable absorbent insert according to claim 1, wherein the insert has a rectangular shape.

25. A wearable absorbent article comprising a reusable outer shell, to be worn by a wearer, and a disposable absorbent insert according to claim 1.
